(19) United States
(12) Patent Application Publication
(10) Pub. No.: US 2026/0145861 A1
(43) Pub. Date: May 28, 2026

(54) DEVICES, SYSTEMS AND METHODS FOR PROCESSING FEEDING DATA

(71) Applicant: Warmer Products, LLC, Dover, DE (US)

(72) Inventor: Anthony M. Souris, Phoenix, AZ (US)

(73) Assignee: Warmer Products, LLC, Dover, DE (US)

(21) Appl. No.: 18/958,997

(22) Filed: Nov. 25, 2024

(51) Int. Cl. B65D 81/34; G16H 20/60; H04B 5/40

(52) U.S. Cl. CPC B65D 81/34; G16H 20/60; H04B 5/40

(57) ABSTRACT

A feeding system comprising a container for storing a meal, the container having a communication tag preloaded with container data comprising a unique container ID, a container type and/or a container weight. The communication tag is configured to store, receive and transmit container data and child related data associated with a unique child profile. The system comprises a peripheral device configured to store, receive and transmit the container data and the child related data, wherein the communication tag and the peripheral device communicate with each other. The child related data may include caregiver feeding instructions associated with a unique child profile. The system may include a weight sensor to calculate an amount of the meal in the container which is transmitted and stored in the communication tag. The system may include a container warmer and the communication tag communicates warming instructions to the container warmer for warming a meal.

### TECHNICAL FIELD

The present disclosure relates generally to various devices, systems and methods for processing feeding data.

### BACKGROUND

Feeding a bottle-fed baby can be a difficult and time-consuming process for parents and caregivers. The process can include, among other things, managing breast milk expiration dates, maintaining breast milk inventory, preparing baby bottles, communicating feeding instructions, choosing the right warming temperature, and tracking feeding activities across different caregivers. Although there have been improvements to baby feeding accessories and methods for tracking the baby feeding process, they have drawbacks including limitations on the ease of collection, management and the sharing of information across accessories and tracking applications.

For example, calculating the volume of milk in a breast milk bag can be difficult because breast milk bags are flexible and can easily change shape as the milk is poured into the bag, making it difficult, if not nearly impossible, to accurately measure the volume of liquid in the bag. Nonetheless, parents and caregivers need this information to track the quantity of breast milk in storage. They also need this information to determine which bag to use (e.g., based on its volume, age, etc.) depending on how hungry the baby might be.

Furthermore, writing breast milk information onto a breast milk bag (e.g., date, volume, etc.) can be difficult because of the flexibility of the bag and because the bag may be wet, causing the ink to smear, or a pen suitable for writing on a bag may not be readily available. It can also be difficult to write on a bag in low light conditions such as when a mom pumps in the middle of the night. Writing the pump date on a bag is a critical step in managing when breast milk should no longer be used.

Moreover, calculating the volume of liquids in a baby bottle itself can be difficult because the volume markers on the bottle (e.g., gradients of 1 oz., 2 oz., etc.) may be hard to see (e.g., because they are clear or translucent), especially in low light conditions such as when parents feed their baby in the middle of the night. Similarly, the angle at which one views the bottle may also change the perceived volume of liquid in the bottle. Parents and caregivers need this information when preparing a powdered formula bottle because an incorrect ratio of water to formula could have health impacts on a baby.

Additionally, bags can get lost because they are often stored across different locations including the refrigerator, freezer or at a remote caregiver location such as a daycare. This is especially important because it can be difficult to manage a bag's expiration date and because expiration dates may vary depending on the location. For example, the expiration date in a refrigerator is generally shorter than the expiration date in a freezer. Similarly, parents may not always know how many bags have been consumed at a caregiver or how much milk remains in a partially used bag. Not all caregivers have an easy or convenient way to share this information with parents, especially in real time. Likewise, it is not easy to provide caregivers with feeding instructions especially if those caregivers care for multiple babies.

A variety of baby feeding and tracking applications (or “apps”) have been developed and are becoming more popular with parents because they have aspirations of allowing the parents and caregivers to more easily track and manage baby feeding activities and breast milk bag inventory. However, existing apps generally fail to address the issues discussed above. For example, most apps require manual input of data, which can be cumbersome to use to track pumping and feeding activities. It is also cumbersome for caregivers trying to access the app, particularly if the app is not already installed on the caregiver's phone or device.

Accordingly, apparatus, systems and methods for making food storage and bottle feeding easier, smarter and more informative by automating how baby feeding accessories collect, manage and share information, for example through improved feeding and tracking software apps and related data collection devices, are desirable.

### SUMMARY

The present disclosure provides devices, systems and methods for processing feeding data, including a feeding system comprising a container for storing a meal, the container having a communication tag connected thereto, wherein the communication tag may be preloaded with container data comprising a unique container ID, a container type and/or a container weight. The communication tag may be further configured to store, receive and transmit container data and child related data associated with a unique child profile. The system may further comprise a peripheral device configured to store, receive and transmit the container data and the child related data, wherein the communication tag and the peripheral device communicate with each other.

In accordance with various aspects of the present disclosure, the child related data may comprise a child name, a meal type, a meal weight, a storage location, a feeding date, a feeding time, a feeding amount, an expiration date, an expiration time, a heating temperature, a last known meal temperature, and/or a link to the unique child profile. In some embodiments, the child related data comprises caregiver feeding instructions associated with the unique child profile transmitted to the peripheral device.

In accordance with various aspects of the present disclosure, the feeding system may include a weight sensor. When the container is placed on the weight sensor, the weight sensor communicates with the communication tag to determine the type and the container weight to calculate an amount of the meal in the container which is transmitted and stored.

In accordance with various aspects of the present disclosure, the feeding system may include a container warmer. When the container is placed proximate the container warmer, the communication tag communicates warming instructions to the container warmer for warming the meal.

### BRIEF DESCRIPTION OF THE DRAWINGS

The accompanying drawings are included to provide a further understanding of the disclosure and are incorporated in and constitute a part of this specification, illustrate embodiments of the disclosure, and together with the description serve to explain the principles of the disclosure, wherein:

FIG. 1 illustrates a container for storing a meal having a communication tag connected thereto, wherein the communication tag is configured to store, receive and transmit data to a peripheral device in accordance with the present disclosure.

FIG. 2 illustrates a bottle having a communication tag connected thereto in accordance with the present disclosure.

FIG. 3 illustrates a breast milk bag having a communication tag connected thereto in accordance with the present disclosure.

FIG. 4 illustrates a baby bottle having a communication tag connected thereto placed on a weight sensor in accordance with the present disclosure.

FIG. 5 illustrates a breast milk bag having a communication tag connected thereto placed on a weight sensor in accordance with the present disclosure.

FIG. 6 illustrates a baby bottle having a communication tag connected thereto on a container warmer in accordance with the present disclosure.

FIG. 7 illustrates containers having communication tags connected thereto in communication with container warmers, weight sensors, refrigeration devices and liquid dispensers in accordance with the present disclosure.

FIG. 8 illustrates the communication across various containers having communication tags connected thereto in communication with various peripheral devices, including container warmers, weight sensors, and refrigeration devices, communicating with various applications for use by various caregivers in accordance with the present disclosure.

### DETAILED DESCRIPTION OF THE ILLUSTRATED EMBODIMENTS

Persons skilled in the art will readily appreciate that various aspects of the present disclosure can be realized by any number of methods and systems configured to perform the intended functions. Stated differently, other methods and systems can be incorporated herein to perform the intended functions. It should also be noted that the accompanying drawing figures referred to herein are not all drawn to scale but may be exaggerated to illustrate various aspects of the present disclosure, and in that regard, the drawing figures should not be construed as limiting. Finally, although the present disclosure can be described in connection with various principles and beliefs, the present disclosure should not be bound by theory.

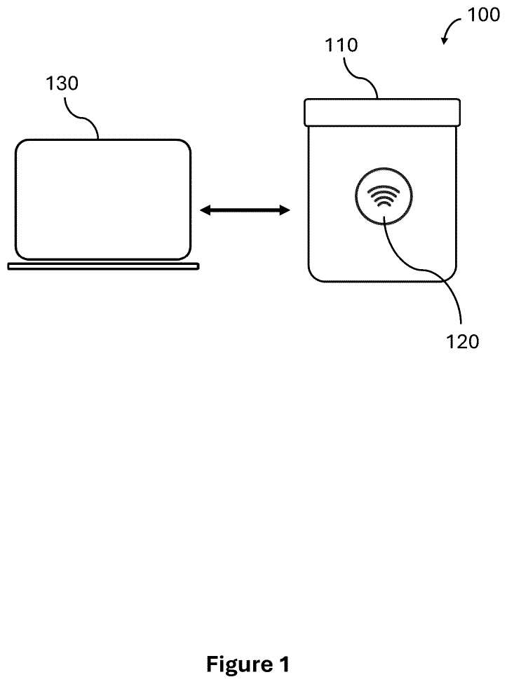

In general, the present disclosure provides devices, systems and methods for processing feeding data and, with reference to FIG. 1, including a feeding system comprising a container 110 for storing a meal, the container 110 having a communication tag 120 connected thereto, wherein the communication tag 120 may be preloaded with container data comprising a unique container ID, a container type and/or a container weight. The communication tag 120 may be further configured to store, receive and transmit container data and child related data associated with a unique child profile. In accordance with various aspects of the present disclosure, the system may further comprise a peripheral device 130 configured to store, receive and transmit the container data and the child related data, wherein the communication tag 120 and the peripheral device 130 communicate with each other.

In accordance with various aspects of the present disclosure and as used herein, “meal” means any type of nourishment or sustenance that can be fed to a human, mammal, or other animal. For example, a “meal” as used herein may include, among other things, a food product such as a pureed fruit, vegetable, meat, soup or similar product, breast milk, baby formula, or even water.

Additionally, in accordance with various aspects of the present disclosure and as used herein, “child” means any person that might need assistance from a caregiver (defined below) such an infant or baby, a toddler, a more traditional meaning of child (persons prior to puberty but older than an infant or toddler), as well as young adults, adults and the elderly that may need assistance with feeding, whether due to illness or other physical or mental incapacity.

Additionally, in accordance with various aspects of the present disclosure and as used herein, “caregiver” means any person who provides care to a child as defined above. For example, caregivers may include parents and grandparents, siblings, babysitters, teachers, nurses and doctors, and any other person that provides care to the child, for example, using the feeding systems disclosed herein.

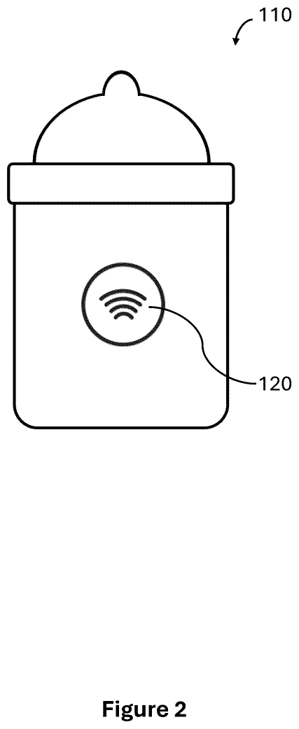

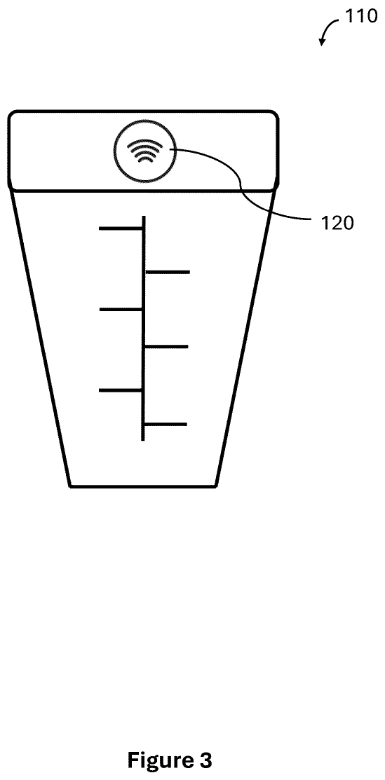

In accordance with various aspects of the present disclosure, the container 110 may be any vessel, now known or as yet known, configured to contain a meal as defined herein. For example, in accordance with various non-limiting embodiments, the container 110 may be a baby bottle (FIG. 2), a breast milk bag (FIG. 3), or other containers such as jars.

In accordance with various aspects of the present disclosure, the child related data may comprise any data that may be used in connection with the feeding system and related aspects of the same disclosed herein or otherwise useful to a caregiver. For example, among other things, child related data may include a child's name, a meal type, a meal weight, a storage location (the location where in the storage container 110 may be found), a feeding date, a feeding time, a feeding amount, an expiration date, an expiration time, a heating temperature, a last known meal temperature, and/or a link to the unique child profile.

By way of example, and as described in more detail below, because storage containers 110 can get lost because they are often stored across different locations including the refrigerator, freezer or at a remote caregiver location such as a daycare and because expiration dates may vary depending on the location, in accordance with various aspects of the present disclosure, the weight sensor can accurately calculate and automatically save an expected expiration date onto the storage container 110 itself and, for example, into a baby tracking app on a cellphone. The app, the weight sensor and other components in accordance with the present disclosure may display which storage containers 110 are about to expire and where to find these storage containers 110 (e.g., refrigerator, freezer, etc.). Each storage containers 110 may be labeled with a unique identifier (e.g., number and/or alpha character) allowing caregivers to find the storage containers 110 faster.

Furthermore, and with specific reference to breast milk bags and as noted above, writing breast milk information onto a breast milk bag (e.g., date, volume, etc.) can be difficult because the bag may be wet, causing the ink to smear, or because a pen suitable for writing on a bag may not be readily available. It can also be difficult to write on a bag in low light conditions such as when a mom pumps in the middle of the night. Writing the pump date on a bag is a critical step in managing when breast milk should no longer be used.

In accordance with various aspects of the present disclosure, and with continuing reference specifically to the breast milk bags, communication tags 120 on the breast milk bags may be programmed with local data so that caregivers do not need to access remote data such as data from a server, eliminating the need to write on the bag. For example, the communication tag 120 on the breast milk bag can be saved locally directly on the bag with data such as the heating temperature (e.g., 98° F.) so that caregiver cellphones, or other peripheral devices 130 with communication capability that caregivers may be using, know to what temperature to warm the bag.

Additionally, and as noted above, calculating the volume of liquids in a baby bottle itself can be difficult because the volume markers on the bottle (e.g., gradients of 1 oz., 2 oz., etc.) may be hard to see (e.g., because they are clear or translucent), especially in low light conditions such as when parents feed their baby in the middle of the night. Similarly, the angle at which one views the bottle may also change the perceived volume of liquid in the bottle. Moreover, parents and caregivers need this information when preparing a powdered formula bottle because an incorrect ratio of water to formula could have health impacts on a baby. Thus, in accordance with various aspects of the present disclosure and as described in more detail herein, by using a weight sensor to calculate the volume of liquids in a baby bottle more accurately, parents and caregivers can ensure a correct ratio of water and formula.

In accordance with various aspects of the present disclosure, the child related data may comprise caregiver feeding instructions associated with the unique child profile transmitted to the peripheral device 130 in the form of a cellphone. In accordance with various aspects of the present disclosure, the child related data may be stored in a variety of types of external databases, such as a remote child related data storage device, including cloud storage and web servers, local computer devices (e.g., person computers), networked attached storage (NAS) and similar backup storage, mobile communication devices (e.g., cellphones, tablets, lap top computers), and various mobile applications.

As one skilled in the art will appreciate, the databases may comprise a single physical database, multiple physical database, etc. For example, the database may be an Internet server with a plurality of web servers that service data requests and acquire and share data.

Additionally, various security and authentication mechanism may be provided, including any hardware and/or software suitably configured to receive authentication credentials, encrypt and decrypt credentials, authenticate credentials, and grant access rights according to privileges (e.g., pre-defined privileges) attached to the credentials. The authentication mechanisms may grant varying degrees of application and data level access to caregivers based on information stored within the various components of the feeding system 100.

In accordance with various aspects of the present disclosure, a link to the unique baby profile may provide caregivers with access to specific feeding instructions programmed by the parent, including, for example, the recommended quantity to feed a baby or what temperature to heat a bottle too. The link may be wirelessly accessed by the caregiver through an approved device such as a cellphone or other peripheral device.

Additionally, with specific reference to breast milk bags in accordance with various aspects of the present disclosure, including a unique baby profile in the baby tracking app allows caregivers (such as daycares) to save information related to when a breast milk bag has been partially or fully consumed. The same may also apply to other storage containers 110. The information saved by the caregiver is automatically updated in the baby tracking app so that parents know when more milk (or formula, etc.) is needed at the daycare.

In accordance with various aspects of the present disclosure, the communication tag 120 may comprise any device capable of storing data such as that contemplated herein and, preferably, wirelessly communicating (transmitting and/or receiving) such data between the communication tag 120 and another device, such as a peripheral device 130 contemplated herein. For example, a communication tag 120 contemplated herein may include a conventional Near Field Communication (NFC) tag.

Some communication tags 120, such as NFC tags, may be “peel and stick” stickers which may be easily attached to devices such as the storage container 110, including containers such as breast milk bags which may not be as rigid as other containers such as bottles.

Moreover, communication tags 120 may also be removable to the extent it may be desirable to remove and re-attach or replace the communication tags 120. Communication tags 120 may be designed to withstand extreme temperatures such as 32° F. and below such as when a meal (e.g., breast milk) is in a freezer and up to and beyond 212° F. such as when warming meal (e.g., pre-made baby formula) in a container warmer such as a steam bottle warmer. The communication tag 120 may also be designed to withstand condensation such as when defrosting a breast milk bag in the refrigerator and can be submerged in water such as when defrosting breast milk bags in a warm water bath.

Additionally, other types of communication tags, now known or as yet unknown, may substituted with conventional NFC tags and still fall within the scope of the present disclosure to the extent they are able to store the data contemplated and wirelessly communicate the data between the communication tag 120 and the peripheral device 130, including via communication technologies such as nanoscale, near-me, Wi-Fi networks, Bluetooth®, ethernet and local area networks (LAN), wide area networks (WAN), cloud networks, cellular and other Internet networks and the like.

In accordance with various aspects of the present disclosure, the communication tag 120 may include various visual features, for example, to help identify a specific container 110 to which the communication tag 120 is attached. For example, a visual feature may include, but not limited to, numeric, alpha, alphanumeric and color variations.

As noted above, the communication tag 120 may be preloaded with container data such as a unique container identification (ID), a container type and/or a container weight.

The unique container ID generally includes a datum which identifies a specific, unique container to which the communication tag 120 is attached in order to distinguish it from other containers. For example, the unique container ID may be a serial number or similar code that is not associated with any other particular container. The container type data generally describes the type of container to which the communication tag 120 is attached, such as a baby bottle, breast milk bag or other storage container. The container weight data describes the weight of the specific container to which the communication tag 120 is attached. Multiple different containers may be of the same type (e.g., a breast milk bag) or have the same wight, but the multiple different containers would not share a unique container ID.

As mentioned above, in accordance with various aspects of the present disclosure, a weight of the container 110 may be useful for determining important aspects related to feeding a child, such as determining the volume of the meal in the container 110, as well as how long and to what temperature the meal should be heated.

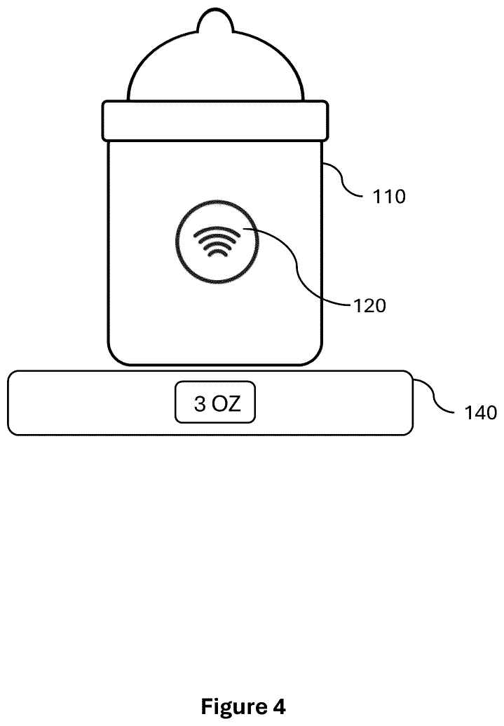

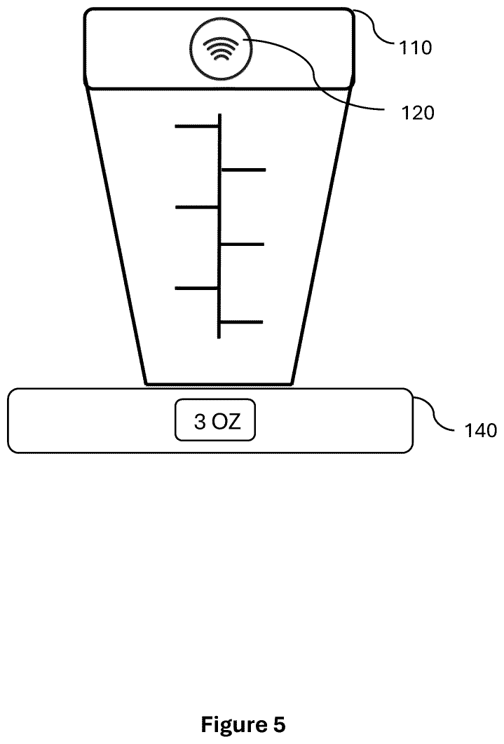

For example, with reference now to FIGS. 4 and 5, the feeding system may further comprise a weight sensor 140. In this regard, a meal (breast milk, formula, etc.) is typically placed in a container 110 and maybe stored for later use, for example in a refrigerator, freezer or the like. As described in more detail below, details relating to storage may be processed in accordance with various aspects of the feeding system 100.

When a caregiver is ready to start the process of feeding a child, the container 110 may be placed on the weight sensor 140. The weight sensor 140 communicates with the communication tag 120 to determine the type of container (e.g., bottle or breast milk bag) and the container weight, the type of meal in the container 110 and/or similar data (e.g., the density of the meal in the container 110), and calculates an amount of the meal in the container 110 by measuring the combined weight of the container 110 and the meal, and subtracting the weight of the container 110 from the combined weight to determine the weight of the meal, and using the weight of the meal to identify the amount of the meal in the container 110.

Thus, in accordance with various aspects of the present disclosure, the weight sensor can accurately calculate the mass and/or volume of the meal in the container 110 and automatically save this information, along with data such as pump date, container 110 location, both geographically such as at home or at a caregiver (e.g., daycare) or within a location (e.g., a refrigerator or freezer), along with expiration dates, onto the bag itself and into a baby tracking app which is faster and easier than handwriting this information onto a bag. This information may be wireless and digitally saved, which means that pens are not required for handwritten notes. This likewise makes it faster and easier than manually opening and manually typing data into an app. By saving the information onto the bag itself, any authorized parent or caregiver can access this information quickly without needing to download the app or manually search for the information on a mobile app server.

Additionally, by way of example, because calculating the volume of milk (or other fluid or meal) in a breast milk bag can be difficult because breast milk bags are flexible and can easily change shape as the milk is poured into the bag, in accordance with various aspects of the present disclosure, using a weight sensor to calculate the quantity of milk in the breast milk bag more accurately determines the volume of milk, for example, within 0.25 oz., regardless of the bag shape or size.

Once determined as described above, the amount of the meal can then be transmitted and stored on the communication tag, another peripheral device (such as cellphone or other device as described in more detail below), and/or another a data storage device.

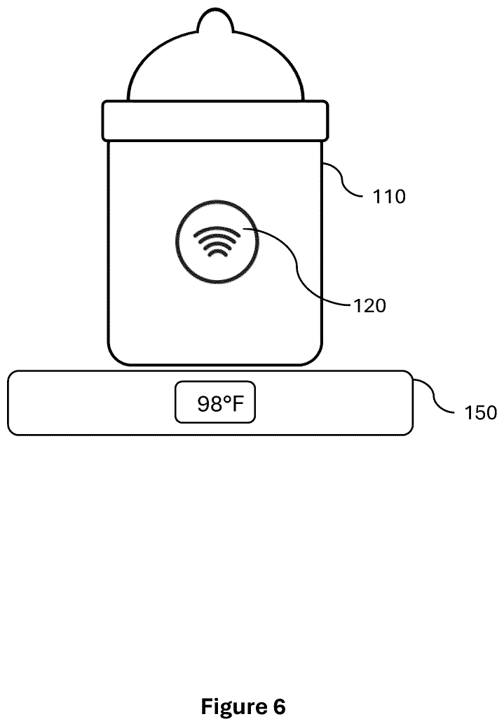

With reference now to FIG. 6, the feeding system 100 may further comprise a container warmer 150. The container warmer 150 is a device configured to warm the contents of the container 110, for example, by placement of the container 110 within the container warmer 150 or by otherwise placing the container 110 in proximity to the container warmer 150 in such a way as to facilitate heat transfer from the container warmer 150 to the container 110. Exemplary technologies for generating heat from the container warmer 150 include induction heaters, film heaters, silicone heaters, air heaters, immersion heaters, cartridge heaters and tubular heaters, as well as other heating technologies now known or as yet unknown.

In accordance with various aspects of the present disclosure, the type and weight of the meal determined as described above can be used to determine the parameters for warming the meal, including the heating temperature, a meal target temperature, a temperature of the warmer and how long the warmer should be activated to warm the meal. For example, when the container 110 is placed proximate the container warmer 150, the communication tag 120 can communicate the data above and/or warming instructions (e.g., heating temperature, target temperature, duration of heating, etc.) to the container warmer 150 for warming the meal. By way of example, a baby bottle or breast milk container 110 with a communication tag 120 can be programmed with instructions to heat the meal to 98° F. and can thus transmit a temperature set point to the container warmer 150 using, for example, NFC read/write capabilities. The container warmer 150 then automatically sets the warming temperature to 98° F. The container warmer 150 can transmit an expiration timestamp back to the communication tag 120 so that any caregiver accessing the data on the communication tag 120 will know when the meal in the container 110 expires (e.g., 2 hours after the container has been warmed).

In accordance with various aspects of the present disclosure, the weight sensor and the container warmer can be the same device. Stated otherwise, the container 110 with the meal is placed on the combined weight sensor and warmer, which in turn calculates the amount of the meal in the container 110, records the desired weight sensor related data as described above and then uses the data as described herein with the container warmer 150 above without the need to move the container 110 from one device to another.

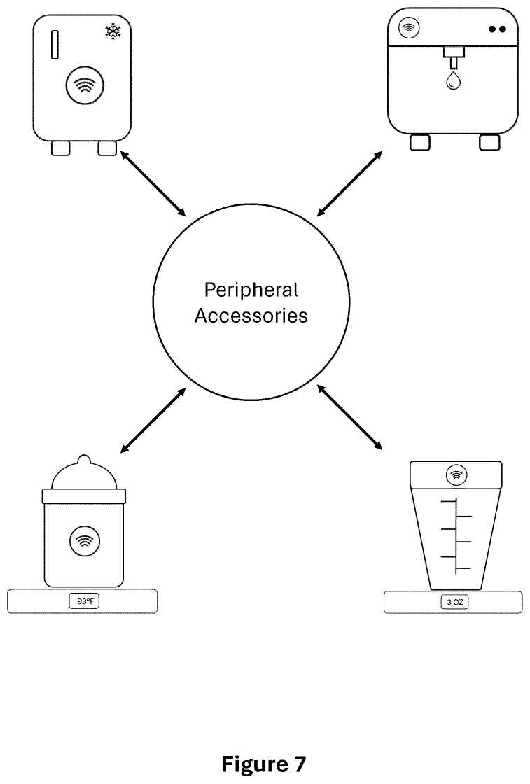

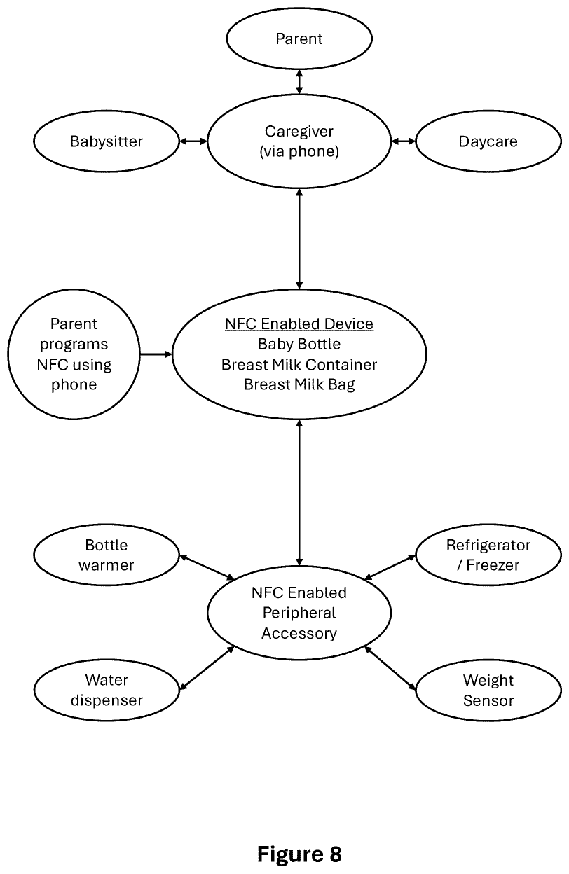

In accordance with various aspects of the present disclosure, instead of or in addition to a weight sensor or container warmer, with reference to FIGS. 7 and 8, the peripheral device 130 (or additional peripheral device 130) may be a personal electronic device such as a cellphone or tablet, personal computer, an appliance such as refrigerator, freezer or similar cooling device, water dispenser, or other peripheral device enabled for communication with the communication tag 120 and/or other peripheral devices, all of which may communicate with and between one other and various caregivers (parents, babysitters, daycare staff, and the like). For example, the peripheral device 130 may be a fluid dispenser (e.g., for water, formula, etc.) which communicates with the communication tag 120, the communication tag 120 providing dispensing instructions, such as the amount of fluid to be dispensed by the fluid dispenser. By way of another example, where the peripheral device 130 is a cooling device to keep the meal cold during storage, the communication tag 120 may communicate inventory instructions and other related data to the cooling device.

In accordance with various aspects of the present disclosure, the peripheral device 130, whether it is a weight sensor, container warmer or other peripheral device 130, may comprise a display such as conventional video screen (e.g., LED, LCD, or the like) for reviewing the data associated with the feeding system 100, the specific peripheral device having the display screen, or data related to the other peripheral devices. There may also be visual indicators (e.g., LEDs) indicating the status of the peripheral device 130, including, but not limited to power ON/OFF, heating, cooling, or the like. Such peripheral devices 130 may also include mechanisms for inputting child related data, including buttons, keyboards, virtual keyboards (e.g., integrated with the display screen), keyboard ports, and the like.

Finally, it will be apparent to those skilled in the art that various modifications and variations can be made in the present disclosure without departing from the spirit or scope of the disclosure. Thus, it is intended that the present disclosure cover the modifications and variations of this disclosure provided they come within the scope of the appended claims and their equivalents.

Likewise, numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications may be made, especially in matters of structure, deposition materials, elements, components, shape, size, and arrangement of parts including combinations within the principles of the disclosure, to the full extent indicated by the broad, general meaning of the terms in which the appended claims are expressed. To the extent that these various modifications do not depart from the spirit and scope of the appended claims, they are intended to be encompassed therein.

I claim:

1. A feeding system comprising,
a container for storing a meal, the container having a communication tag connected thereto, wherein the communication tag is configured to store, wirelessly receive and wirelessly transmit container data and child related data associated with a unique child profile;
a peripheral device configured to store, wirelessly receive and wirelessly transmit the container data and the child related data; and
wherein the communication tag and the peripheral device wirelessly communicate with each other.

2. The feeding system of claim 1, wherein the communication tag is preloaded with container data comprising at least one of a unique container ID, a container type and a container weight.

3. The feeding system of claim 1, wherein the child related data further comprises at least one of a child name, a meal type, a meal weight, a storage location, a feeding date, a feeding time, a feeding amount, an expiration date, an expiration time, a heating temperature, a last known meal temperature, and a link to the unique child profile.

4. The feeding system of claim 1, further comprising a weight sensor, wherein when the container is placed on the weight sensor, the weight sensor communicates with the communication tag to determine the container type and the container weight to calculate an amount of the meal in the container, and wherein the amount of the meal is transmitted and stored in at least one of the communication tag, the peripheral device, and a data storage device.

5. The feeding system of claim 4, further comprising a container warmer, wherein the container warmer is at least one of an induction heater, a film heater, a silicone heater, an air heater, an immersion heater, a cartridge heater and a tubular heater, and wherein when the container is placed proximate the container warmer, the communication tag communicates warming instructions to the container warmer for warming the meal.

6. The feeding system of claim 5, wherein the weight sensor and the container warmer are the same device.

7. The feeding system of claim 1, wherein the child related data comprises caregiver feeding instructions associated with the unique child profile transmitted to the peripheral device.

8. The feeding system of claim 1, wherein the peripheral device is a cellphone.

9. The feeding system of claim 1, wherein the communication tag is a Near Field Communication tag.

10. The feeding system of claim 1, wherein the child related data is stored in at least one of an external database and a mobile application.

11. The feeding system of claim 1, wherein the communication tag is removable.

12. The feeding system of claim 1, wherein the container is at least one of a bottle and a bag.

13. The feeding system of claim 1, wherein the meal is at least one of water, a food product, breast milk and baby formula.

14. The feeding system of claim 1, wherein the peripheral device is a fluid dispenser and wherein the communication tag communicates dispensing instructions to the fluid dispenser.

15. The feeding system of claim 1, wherein the peripheral device is a cooling device to keep the meal cold during storage, wherein the communication tag communicates inventory instructions to the cooling device.

16. A feeding system comprising,
a container for storing a meal, the container having a communication tag connected thereto, wherein the communication tag is configured to store, wirelessly receive and wirelessly transmit container data and child related data associated with a unique child profile;
a mobile application for installation on a personal electronic device;
a remote child related data storage;
at least one peripheral device configured to store, wirelessly receive and wirelessly transmit the container data and the child related data; and
wherein the communication tag, the mobile application, and the at least one peripheral device wirelessly communicate with each other.

17. The feeding system of claim 16, wherein the communication tag is preloaded with container data comprising at least one of a unique container ID, a container type and a container weight.

18. The feeding system of claim 16, wherein the peripheral device is a weight sensor.

19. The feeding system of claim 16, wherein the peripheral device is a container warmer comprising at least one of an induction heater, a film heater, a silicone heater, an air heater, an immersion heater, a cartridge heater and a tubular heater.

20. The feeding system of claim 16, wherein when the container is placed on the weight sensor, the weight sensor communicates with the communication tag to determine the container type and the container weight to calculate an amount of the meal in the container, and wherein the amount of the meal is transmitted and stored in at least one of the communication tag, the peripheral device, and a data storage device.

21. The feeding system of claim 18, further comprising a container warmer, wherein when the container is placed proximate the container warmer, the communication tag communicates warming instructions to the container warmer for warming the meal.

22. The feeding system of claim 16, wherein the peripheral device is a weight sensor and a container warmer.

23. The feeding system of claim 16, wherein the child related data comprises caregiver feeding instructions associated with the unique child profile and are transmitted to the peripheral device.

24. The feeding system of claim 16, wherein the remote child related data storage is a server.

25. The feeding system of claim 16, wherein the communication tag is a Near Field Communication tag.

26. The feeding system of claim 16, wherein the communication tag is removable.

27. The feeding system of claim 16, wherein the container is at least one of a bottle and a bag.

28. The feeding system of claim 16, wherein the meal is at least one of water, a food product, breast milk and baby formula.

29. The feeding system of claim 16, wherein the at least one peripheral device is a fluid dispenser and wherein the communication tag communicates dispensing instructions to the fluid dispenser.

30. The feeding system of claim 16, wherein the peripheral device is a cooling device to keep the meal cold during storage, wherein the communication tag communicates inventory instructions to the cooling device.
